(19) United States
(12) Patent Application Publication
(10) Pub. No.: US 2017/0115278 A1
(43) Pub. Date: Apr. 27, 2017

(54) USEFUL PROCESS FOR SCREENING IMMUNE RESPONSE MODIFIER

(71) Applicants: Yong Chi Lin, Taipei (TW); Po Wen Lin, Taipei (TW); Po Yu Lin, Taipei (TW); Amy Huimeei Lo, Brea, CA (US); Jing Meei Lin, Taipei (TW)

(72) Inventors: Yong Chi Lin, Taipei (TW); Po Wen Lin, Taipei (TW); Po Yu Lin, Taipei (TW); Amy Huimeei Lo, Brea, CA (US); Jing Meei Lin, Taipei (TW)

(21) Appl. No.: 15/356,243

(22) Filed: Nov. 18, 2016

Related U.S. Application Data
(63) Continuation-in-part of application No. 14/107,086, filed on Dec. 16, 2013.

(51) Int. Cl. G01N 33/50; A61K 31/664

(52) U.S. Cl. CPC G01N 33/5055; A61K 31/664

(57) ABSTRACT

A useful HIV remedy process consists of 4 elements: guinea pig or mouse peritoneal derived adherent macrophages/monocytes as effector cells; cyclophosphamide as an immuno suppressor; chicken RBC as target cells; and the anti-HIV₁ agent candidate to be examined.

Immunovir and components were isolated from Pyrus serotina Rehder and other species of Rosaceae by column chromatography.

### STATEMENT REGARDING FEDERALLY SPONSORED RESEARCH OR DEVELOPMENT

N/A

### REFERENCE TO SEQUENTIAL LISTING, A TABLE, OR A COMPUTER PROGRAM LISTING APPENDIX SUBMITTED ON A COMPACT DISC

N/A

### CROSS-REFERENCE TO RELATED APPLICATIONS & CONTINUITY DATA

N/A

### BACKGROUND OF THE INVENTION

AIDS is extremely difficult to cure for many reasons. First, nucleoside analogue reverse transcriptase inhibitor (NART I) and non-NART I or protease inhibitor are competitive inhibitors. They do not inhibit human immunodeficiency virus (HIV) replication completely, and can induce persistent infecting cells, resting cells, and drug fasting easily.

Second, HIV is a highly variable virus. Isolating the virus from different organs of the same patient would not result in identical samples of the virus.

Third, HIV has a lysis effect on CD+cells. Its constituents, particularly surface antigens, have difficulty signaling Th cells. Thus, an inadequate amount of antibody is produced to activate the antibody-dependent cytotoxic cells (ADCC) to kill the monocytes and macrophages that are persistently infected with the virus.

Therefore, carriers of HIV have consistently low antibody titer, and continuously spread out HIV. Based on the understanding of the HIV mechanism, this agent intends to activate killer cells, which can damage cells persistently infected with HIV. By releasing immature and non-infections virion and surface antigens to promote antibody production, ADCC activity can be activated (and vice versa) to ultimately cure AIDS.

### FIELD OF THE INVENTION

This present invention relates to the field of anti-HIV agents with good curative effects against AIDS.

### SUMMARY OF THE INVENTION

This invention is a low-cost anti-HIV agent with good curative effects against AIDS. Plant ingredients of this agent were obtained via plant harvest, ingredient extraction, refinement, and specification. Such ingredients were used in anti-HIV in vitro tests, anti-AIDS in vivo tests, and adverse effect and safety tests.

The agent has been proven to inhibit HIV replication in vitro and cure SIVmac L28 infection in vivo. It provides good curative effects against AIDS with low adverse effects and is a safe and low-cost anti-HIV and anti-AIDS agent.

### BRIEF DESCRIPTION OF THE DRAWINGS/FIGURES

FIG. 1-1 shows the response of target chicken red blood cells (cRBC) and guinea pig peritoneal derived adherent macrophages culture with R7½G as effector cells (gMφ) to Immunovir A(1) and/or cyclophosphamide.

FIG. 1-2 shows a graphical representation of the experimental system.

FIG. 1-3 shows the effects of immunovir and/or cyclophosphamide on gMφ activity in vitro.

FIG. 2 shows immunovir A, B, C, and D activation of Mφ/Mo in mice.

Table 1 summarizes the effects of Immunovir on killer cell activity in mice.

Table 2 summarizes the effect of Immunovir and Cyclophosphamide on serum hemolysin titer in cRBC immunized mice.

Table 3 summarizes the hemagglutination activity of immunovirs.

### DETAILED DESCRIPTION OF THE INVENTION

### EXPERIMENTS

### Experiment 1

Effect on cellular immunity in vitro:

The effector (killer) cells for this experiment were adherent macrophages (gMφ) derived from guinea pig's peritoneal fluid cultured with R7½G (RPMI 1640 enriched with 7.5% v/v guinea pig serum). Cyclophosphamide (CP) was used as an immunosuppressive agent. Chicken red blood cells (cRBC) as target cells.

The experiment consisted of four series of experiments: gMφ+cRBC, gMφ+Imm+cRBC, gMφ+CP+cRBC, and gMφ+CP+Imm+cRBC, and immunovir (Imm) as immune response modifier.

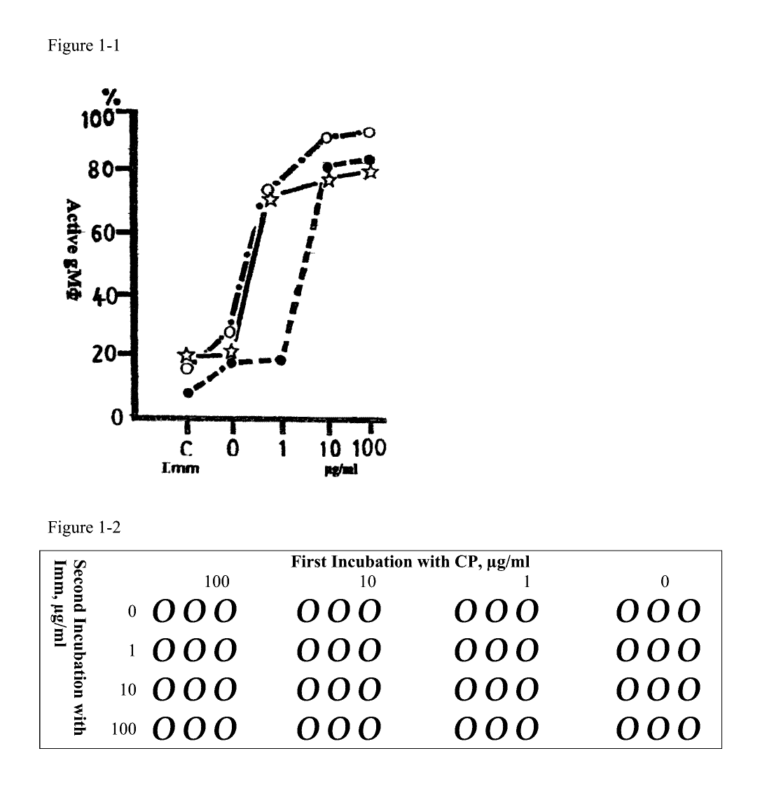

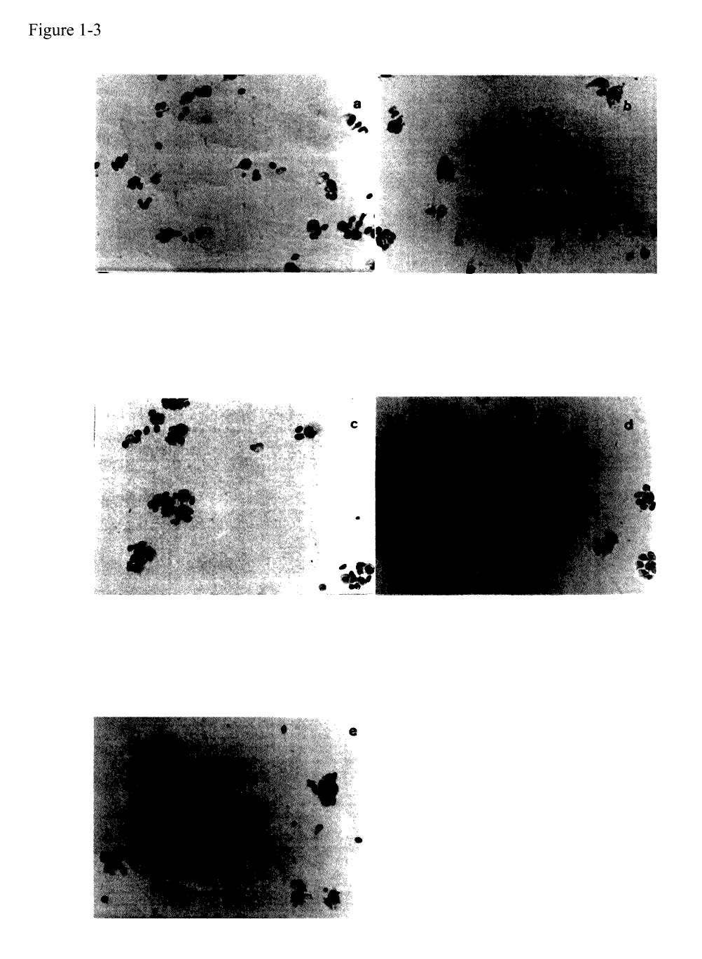

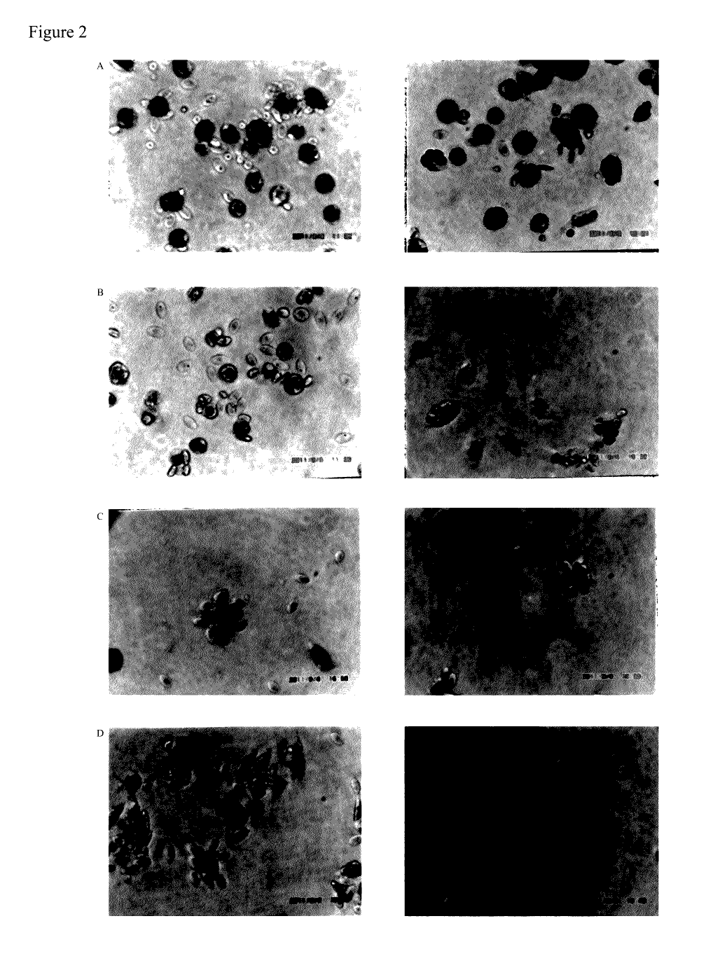

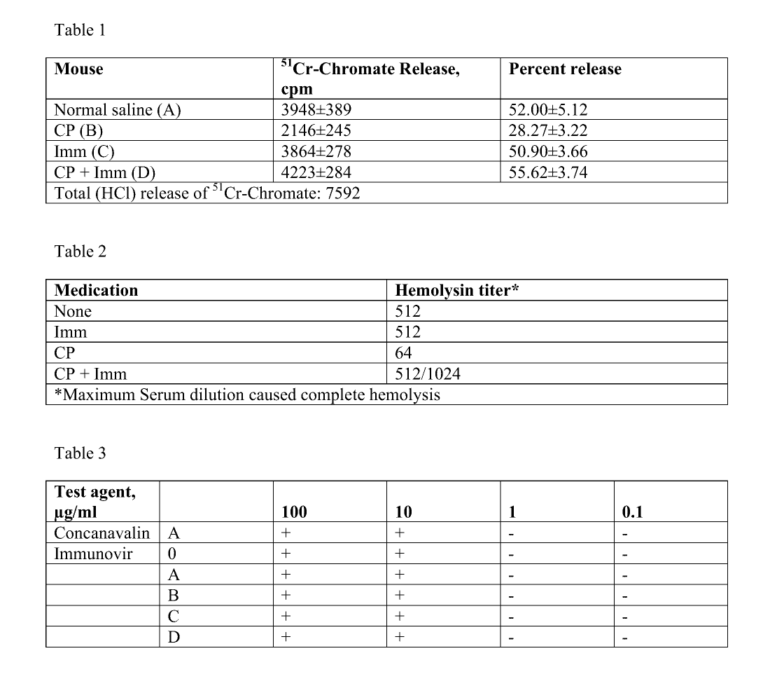

A 200 to 250 g guinea pig was injected with 1 mL of thioglycolate medium, after 20 hours adherent macrophages derived from abdominal cavity (gMφ) were collected by aid of RPMI 1640, suspended in R7½G, then 0.9 mL of solution was pipetted into thirteen Falcon 12-well culture plates. The plates for group 1 and 2 to group 4 were 1 and 3, respectively. See FIG. 1-2. Cyclophosphamide (CP) was added into the wells from group 2 to group 4 so that the final concentration was 1, 10, and 100 ug/mL respectively. In other words, each well was filled with 1 ug/mL CP in group 2, 10 ug/mL in group 3, and 100 ug/mL in group 4. After incubation in a 5% CO2 incubator (35-37° C.), the medium in each well was replaced with fresh R7½G from group 1 (only 1 plate for control) to group 4. Then Imm was added from lane 1 to lane 4 (3 wells per lane) that the final concentration were 0, 0.1, 1, and 10 ug/mL, respectively. That was to say, from group 2 to group 4, lane 1 was a CP control, and lane 1 in plate 1 was gMφ control. After subsequent incubation in 5% CO₂ incubator, medium in each well of plate was replaced with 0.2% cRBC-containing RPMO 1640. cRBCs were sucked out after another two to six hours of incubation and adhered cells (gMφ and attached cRBC) were stained by Liu Stain. The percentage of active gMφ in 300 gMφ or above was calculated under the optical microscope, and the results were shown in FIGS. 1-1, as C and O in the figure indicated that activity of gMφ were unrecoverable with the removal of CP suppression in spite of the second 20-hours incubation, and demonstrated that a 1/10 concentration of immunovir could remove the suppression of CP.

FIG. 1-3 demonstrated that immunovir removed the immuno suppression of CP and increased the killer cell activity of individual gMφ.

FIG. 1-1 also demonstrated that immunovir neutralized 10-fold immuno suppression of CP.

### Experiment 2

Effect of mice macrophages/mononuclear cells activities in vivo.

Mice were injected with cyclophosphamide 200 mg/kg.b.w and 100 mg/kg.b.w via tail vein in the morning of day 1 and day 2, respectively. Two mice of each group were injected with 10 mg/kg.b.w of immunovir (O, mixture), immunovir A, B, C, D, or 20 mg/kg.b.w of AZT via tail vein in the afternoon from day 2 to day 5, respectively. Each mouse's abdominal cavity was injected with 0.5 mL of R7½C in the afternoon of day 5, and mMφ//Mo were collected from each mouse's abdominal cavity with 10 mL of R7½C in the afternoon of day 6. Basal medium rich in deposit cells were taken, and 0.40 mL was pipetted into two wells of flat-bottomed 24-well Falcon culture dish. After incubation with 5% CO₂ for 6 hours, 0.10 mL of cRBC (1%) was added into each well and incubation was carried out for 6 hours or overnight. Then suspended cells, i.e., cRBC, were sucked out, the well was gently washed with 0.5 mL of RPMI 1640, and 0.40 mL of RBMI 1640 and 5 ul of Liu Stain B solution were added again to stain mMφ, whereas cRBCs were unstainable. CP immuno suppression was removed by Immunovir(mix), Immunovir A, B, C, D, etc. but not AZT in mouse's body, and the efficacy of Mφ/Mo activity increased as shown in FIG. 2.

### Experiment 3

The efficacy of immunovir to mononuclear killer cell activity derived from mouse's spleen:

Twenty male BALB/c mice aged 8 weeks were divided into group A, B, C, and D. Mice in group A were injected with 0.20 mL of normal saline intravenously. Group B received cyclophosphamide (CP) 200 mg/kg.b.w and 100 mg/kg.b.w at day 1 and day 2, and subsequently, received normal saline every day. Group C received immunovir 10 mg/kg.b.w every day. Group D received CP as group B and immunovir as Group C. All mice's spleens were excised at day 7 and spleen-derived mononuclear cells were isolated by Ficol-paque centrifugation.

Yac-1 cells (2×10⁶/mL) were labeled with R20C containing 1 uc/mL of ⁵¹Cr-chromate for 60 minutes at 37° C.

Mouse's spleen-derived mononuclear cells (killer cells) (3×10⁶) and ⁵¹Cr-chromate-labeled Yac-1 cells (6×10⁶) were suspended altogether in 1.0 mL of R20C medium and incubated at 37° C. for 150 minutes, centrifuged with 250 g for 10 minutes, then 0.50 mL of supernatant was taken and dried in the bottle.

Radioactivity was measured by liquid scintillation and toluene-PPO-POPOP was used as a scintillant.

The results were shown in Table 1, the radioactivity released was inhibited by CP, but neutralized by 1 mm later.

### Experiment 4

Hemagglutination activity:

Immunovir A, B, C, and D, or concanavalin A with different concentrations were added into 2 mL of normal saline 0.4% v/v cRBC suspension in Kahn tube. The solution was mixed thoroughly and kept at room temperature for 2 hours to observe hema-agglutination. The results were shown in the Table 3. The tested drugs had same activities, and blood cells were firmly agglutinated with concanavalin A and could not be resuspended by shaking, whereas cRBCs agglutinated with immunovir A, B, C, D could be re-suspended evenly by shaking and re-agglutinated. It suggested that the administration of immunovir A, B, C, and D might be injected intravenously without forming a blood clot.

### Experiment 5

Immunovir enhancement of humoral immunity:

Sixteen normal and CP-immunosuppressing mice were used. Four mice of each group was tested with the effect of immunovir (mix) for the ability of hemolysin (antibody) production of cRBC (antigen) after immunization.

At day 1, mice were injected with 0.05 mL of normal saline (NS) suspension of 0.4% v/v cRBC via tail vein.

Group 1 mice were injected with 0.05 mL NS from day 1 to day 5 (normal control)

Group 2 mice were injected with 0.02 mg of immunovir (10 mg/kg b.w,i.v.).

Group 3 mice were injected with 4.0 mg of cyclophamide (200 mg/kg b.w., i.v.) at day 1, 2.0 mg of CP (100 mg/kg b.w.i.v.) at day 2, and N.S. from day 3 to day 5 (Immunoresponse suppression group).

Group 4 mice were injected with CP at day 1 and day 2 as well as group 3, and 0.20 mg of immunovir (Imm) (10 mg/kg b.w, i.v.) from day 1 to day 5 for the efficacy test of Imm to serum hemolysin or antibody titer.

All mice were sacrificed at day 6, and sera were taken for the test on hemolysin (anti-cRBC antibody) titter.

For a flat-bottomed 96-well (8×12 wells) Falcon plate, 100 uL of mice serum (1:8 dilution) were added into the well 1 in lane 1, then a two-fold dilution series was performed down to the well 12. Besides 60 uL of a 0.25% cRBC, and 50 uL normal guinea pig (GP) serum (1:8 dilution) was added into each well as a complement, and the final concentration was 1 unit/mL.

After incubation in 5% CO₂ incubator at 37° C. for 6 hours, hemolysis was observed under the optical microscope. The hemolysin (antibody) titer was defined as the highest dilution of mice serum with complete hemolysis, and the results were shown in Table 2.

What is claimed is:

1. The process for screening immune-response modifiers for possible HIV remedy comprised of an immuno suppressor, effector cells, target cells, and the target candidate.

2. The process of claim 1, whereas the immune suppressor is cyclophosphamide.

3. The process of claim 1, whereas the effector cells is either guinea pig or mouse peritoneal-derived macrophages/monocyte.

4. The process of claim 1, whereas the target cell is chicken red blood cells.
